(19) United States
(12) Patent Application Publication
(10) Pub. No.: US 2021/0244480 A1
(43) Pub. Date: Aug. 12, 2021

(54) Reference Navigation Equipment in Robotic Surgery Assisted by Stereotaxic Navigation of the Organs and Soft Parts of a Patient's Pelvic Area

(71) Applicants: Fundacion Institute de Investigacion Marques de Valdecilla (IDIVAL), Santander (ES); Servico Cantabro de Salud, Santander (ES)

(72) Inventors: Marcos GOMEZ RUIZ, Santander (ES); Alberto CALLEJA IGLESIAS, Santander (ES); Felipe ALONSO HORGA, Santander (ES); Ruben MARTIN LAEZ, Santander (ES)

(21) Appl. No.: 16/973,564

(22) PCT Filed: Jun. 3, 2019
(86) PCT No.: PCT/ES2019/070373

§ 371 Date: Dec. 9, 2020

(30) Foreign Application Priority Data
Jun. 15, 2018 (ES) U201830919

(51) Int. Cl. A61B 34/20; A61B 34/30; A61B 90/14; A61L 31/02; A61L 31/14

(52) U.S. Cl. CPC A61B 34/20; A61B 34/30; A61B 2017/00915; A61L 31/022; A61L 31/14; A61B 90/14

(57) ABSTRACT

The present invention relates to equipment for navigation reference in assisted robotic surgery by means of stereotactic navigation of the organs and soft portions of the pelvic area of a patient, which enables coordinates fixed with respect to a reference frame to be associated to any point of the patient's anatomy in the pelvic area. The equipment comprises a fastening arc (1) with an upper bar (6), two lateral bars (7), joined to the upper bar (6) forming an arc by means of first joining means (8) and second joining means (9) configured to join the lateral bars (7) to a surgical table, two arms (2) joined to the lateral bars (7) by means of third fastening means (10), a first reference device (3) and a second reference device (15) configured to be placed in the surgical instrumentation of the robot.

### OBJECT OF THE INVENTION

The present invention falls within the technical field of medical equipment for surgery. More specifically, it proposes equipment for navigation reference in assisted robotic surgery by means of stereotactic navigation of the organs and soft portions of the pelvic area of a patient. This equipment enables coordinates to be fixed with respect to a reference frame to be attached to any point of the patient's anatomy in the pelvic area.

### BACKGROUND OF THE INVENTION

Until now, surgery has been the only treatment for rectal cancer with demonstrated curative potential. In the evolution of the surgical treatment of this disease there is a key milestone which is the concept introduced by Heald of the need for total mesorectal excision (TME) in order to achieve a suitable oncological treatment. The suitable execution of this surgical technique, which is essential for achieving the correct oncological results, is a large technical need.

The difficulty of the resection of the rectum with TME is determined by the anatomic ratios thereof. The rectum is located in a narrow and angled musculoskeletal tunnel with an abdominal inlet mouth and another outlet one which is the anal sphincter or, surgically, the perineum. Inside that tunnel and closely related to the rectum is a portion of the male genital apparatus, bladder, seminal vesicles, prostate and urethra or the vagina and a bundle of nerves which will provide bowel, genital and urinary functionality.

This gives the rectal resection great technical difficulty due to problems related to visualisation and dissection, and the conventional surgical techniques imply a series of complications derived from this difficulty. This makes it so the surgeon factor is a crucial element in the clinical results. Overcoming these challenges is only possible by applying the best knowledge of the disease, best imaging techniques and, above all, the development of technologies that help the surgeon solve the barriers derived from the anatomic placement of the rectum.

The advances in digital technology are being applied in surgery with great success. Currently, so-called digital operating rooms are already commonly used. In some disciplines, mainly in neurosurgery, 3D images obtained from the patient (computerised axial tomography, magnetic resonance, etc.) have been able to be applied successfully. With a development of imaging integration, by means of navigation software, this 3D technology can be used to change the manner of performing the surgical method. Preoperative planning of the surgery can be determined, performing “virtual surgery” and it also acts as support for taking surgical decisions in real time.

Like a GPS guides a driver along the selected route and updates the position of the vehicle on the map, the image-guided surgery system (navigator) facilitates planning and simulating the surgical approach for the surgeon. Likewise, it enables information to be obtained about the location and progression of the surgical instruments to the lesion and preserves the integrity of the adjacent anatomic structures preventing complications.

In the past few years, the increase in complexity and the need for surgeons to perform minimally invasive surgery have driven the advance of the intraoperative imaging techniques. In this manner, solutions have been developed such as multidimensional equipment, computerised axial tomography and intraoperative resonances. These systems enable the images used by the navigator to be updated at any time during the operation.

However, navigation in anatomic areas that are not stiff (skull, spine) is still a significant challenge. First, it is not always possible to fasten a reference to the anatomy of the patient close to the work area. Second, the organs and soft tissues can be moved, making the navigation lose accuracy. Finally, although there are universal adapters for navigating with certain instruments (aspirators, bipolar forceps, endoscope), the fastening thereof is not always easy and sometimes does not enable them to be used ergonomically.

For these reasons, it is of interest in the field of pelvic surgery, and of rectal cancer in particular, the development of a system which enables image-guided surgery.

From the state of the art, a few reference systems for surgery are known among which the one described in document WO02064042A1 is highlighted. It discloses a patient support system which is maintained in a certain position during pelvic surgeries. It comprises a fastening arc containing two arms, fastening means for fastening to the pelvis and a reference device on surgical equipment and another on the arc.

The most important technical problem associated with this system is that it is configured to be used in specific surgeries in bony portions of the pelvis or of the hip, located on the outside of the pelvis. This system cannot be used to perform robotic surgery of the organs and soft portions of the inside of the abdominal cavity and the pelvis.

In order to achieve navigation in surgery of soft portions of the pelvis we need to fasten to the patient with an accuracy level close to 1 mm. Any other accuracy level could easily give rise to lesions of large vessels or of other organs. The device described in WO02064042A1 does not have a system for securing to the patient which makes it possible to achieve and maintain a sufficient accuracy level, both in the process before the operation as well as during surgery itself.

Likewise, the design of the fastening arc does not enable the securing of the patient to be combined with elements which ensure sufficient space for the work of the arms of the robot on the pelvic or abdominal area.

Other positioning systems are also known, like the one described in document CN105662551A which discloses an auxiliary positioning structure which supports a surgical instrument which is in turn fastened to the body of the patient or the one disclosed in DE10012042C1 which is a device for calibrating the position of the pelvis on an operating table wherein it is locked in the desired position and which comprises rods which emit orientation signals in order to enable surgical navigation.

Documents CN102283689A and U.S. Pat. No. 5,971,997A are also reference systems. For example, document CN102283689A describes a guide device for hip surgery which comprises an arc connected to two bars and an arm for the anchoring thereof to the patient. Furthermore, document U.S. Pat. No. 5,971,997A proposes an apparatus for calibrating a surgical navigator which has an arc for securing the head in a fixed position by means of screws and which comprises a surgical instrument with emitters that, by means of detectors, enable the position relative to the surgical field to be known.

### DESCRIPTION OF THE INVENTION

The present invention describes equipment for navigation reference in assisted robotic surgery by means of stereotactic navigation of the organs and soft portions of the pelvic area (abdominal cavity and pelvis).

The equipment comprises a structure which is modular and adaptable and with at least one fastening arc wherein at least one reference device is in a position visible by the navigator of the robot performing the surgery. At the same time, the structure is configured to be joined to the anatomy of the patient, leaving enough free space for the robotic arm to move suitably and perform all the necessary operations.

The object of the invention is to ensure that any point of the anatomy of the patient in the pelvic area has coordinates fixed with respect to a first reference device joined to the fastening arc. Moreover, the equipment comprises a second reference device configured to couple to the instrumentation of the robot, in a position also visible by the navigator. The combination of these two reference devices makes the navigation of the robot and the triangulation of the position thereof possible.

The geometry of the structure has been designed such that there are no collisions with the mobile elements of the robot during the surgery or with the structure of an O-Arm (scanning device) in the process of obtaining images of the anatomy of the patient by means of a scanner. It is manufactured almost entirely from titanium since it is radiotransparent, in order to prevent interference during the scanning of the patient.

The structure comprises a fastening arc which is joined to the operating table whereon the patient is placed and can be adjusted in inclination, with respect to said table, and in extension, meaning in height, measured with respect to the table.

It also comprises at least two arms with several degrees of freedom with respect to the fastening arc and that are configured to be joined to the patient through two adapters which are fastened to the pelvis. Preferably, said adapters are threaded pins intended to be joined to the iliac crests of the patient. Thus, it prevents the use of additional tools for the fastening of the equipment to the patient which implies an improvement with respect to solutions known from the state of the art.

In the upper portion of the fastening arc the first reference device is joined, assembled such that the position thereof can be varied with respect to the arc and therefore with respect to the table (and to the patient lying thereon).

### DESCRIPTION OF THE DRAWINGS

As a complement to the description provided below, and for the purpose of helping to make the features of the invention more readily understandable, in accordance with a practical preferred embodiment thereof, said description is accompanied by a set of drawings which, by way of illustration and not limitation, represent the following:

FIG. 1 shows a perspective view of the fastening arc, the arms and the first reference device of the equipment for navigation reference in assisted robotic surgery by means of stereotactic navigation of the organs and soft portions of the pelvic area of a patient.

FIG. 2 shows a front view of the fastening arc, the arms and the first reference device.

FIG. 3 shows a top view of the fastening arc, the arms and the first reference device.

FIG. 4 shows a perspective view of the equipment with the components thereof in a position wherein the adapters of the arms are joined to the hip of a patient.

FIG. 5 shows a lateral view of the fastening arc, the arms and the first reference device in the position of FIG. 4 wherein the adapters of the arms are joined to the hip of a patient.

FIG. 6 shows a perspective view of the equipment and of the robotic arm wherein the second reference device is found.

FIG. 7 shows a zoomed-in view of one of the arms with the corresponding adapter thereof and the movements of the elements of said arms have been represented with arrows.

### PREFERRED EMBODIMENT OF THE INVENTION

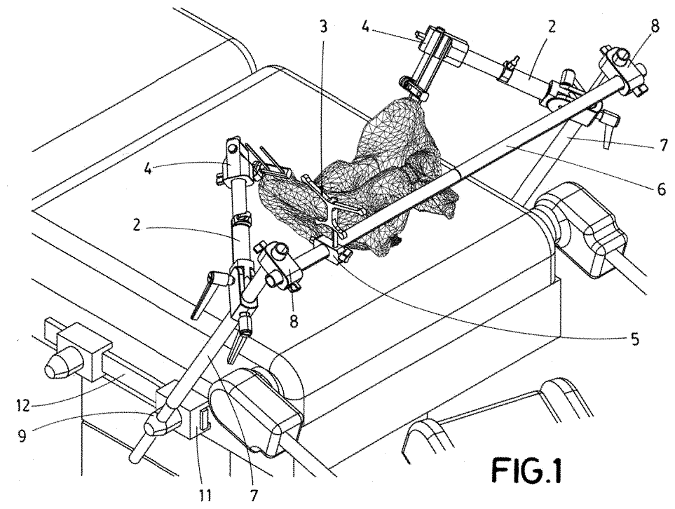

An exemplary embodiment of the invention is described below with help from FIGS. 1 to 7.

More specifically, it proposes equipment for navigation reference in assisted robotic surgery by means of stereotactic navigation of the organs and soft portions of the pelvic area of a patient with surgical instrumentation installed in a robot.

FIG. 1 shows a perspective view of the equipment of the invention joined to a table whereon a patient to be operated on is placed.

The equipment comprises a fastening arc (1) configured to be joined to a surgical table whereon the patient is placed, at least two arms (2) joined to the fastening arc (1) configured to be joined to the patient and reference marks by means of which the navigation of the robot is controlled.

The most important features of the present invention are what enable the correct movement of the robot without there being mishaps with the equipment, which also does not interfere with the scanning equipment and which is completely adaptable to the measurements of the table and of the patient.

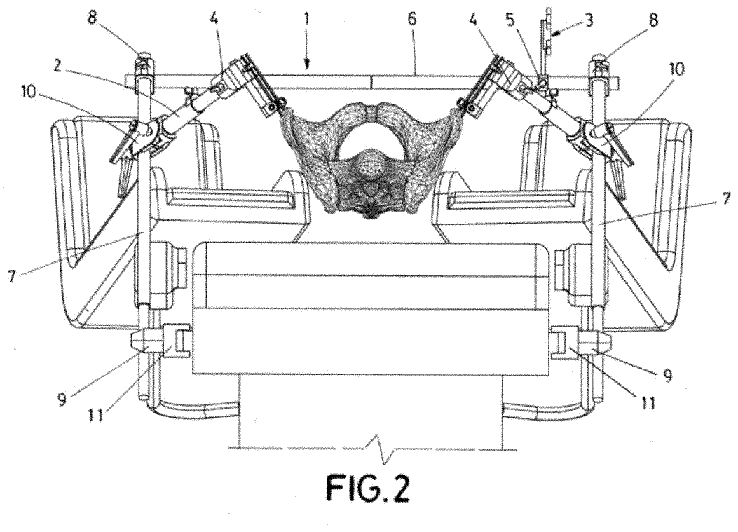

To do so, the fastening arc comprises, as seen in FIG. 2, an upper bar (6) contained in a plane parallel to the surface of the table, two lateral bars (7), joined to the upper bar (6) forming an arc over the table and first joining means (8) and second joining means (9).

The first joining means (8) are linked to the lateral bars (7) and to the upper bar (6) in order to join them. They have the possibility of movement along the upper bar (6) until the fastening thereof in a position adapted to the width of the table wherein the equipment is fastened and they have the possibility of movement along the lateral bars (7) until the fastening thereof in a point corresponding to a height adapted to the position of the pelvis of the patient on the table.

The second joining means (9) are configured to join the lateral bars (7) to the table with the possibility of tilting of the lateral bars (7) around the axial axis of said second joining means (9), in order to regulate the inclination of the lateral bars (7) with respect to the table.

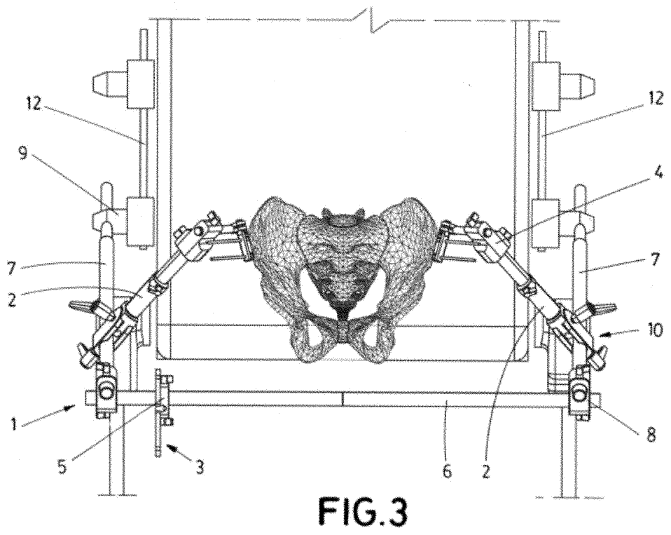

It can also be seen, for example in FIG. 3, that the arms (2) are joined to the lateral bars (7) of the fastening arc (1) by means of third fastening means (10) which have the possibility of movement along the lateral bars (7), with the possibility of rotation with respect to said lateral bars (7). Furthermore, said third fastening means (10) comprise a ball joint configured to enable the tilting of the arms (2) with respect to the longitudinal axis of the lateral bars (7), and each arm comprises an adapter (4) on the free end thereof configured to be temporarily anchored to the patient. The joining of the adapters (4) to the pelvis of the patient just as it stays in the position wherein the equipment is used can be seen for example in FIGS. 1 to 3.

The equipment also comprises a first reference device (3) comprising a mobile part (5) joined to the upper bar (6) with the possibility of movement along said upper bar (6). It also has the possibility of rotation around the upper bar (6) and comprises at least one first reference mark detectable by a navigator of the robot.

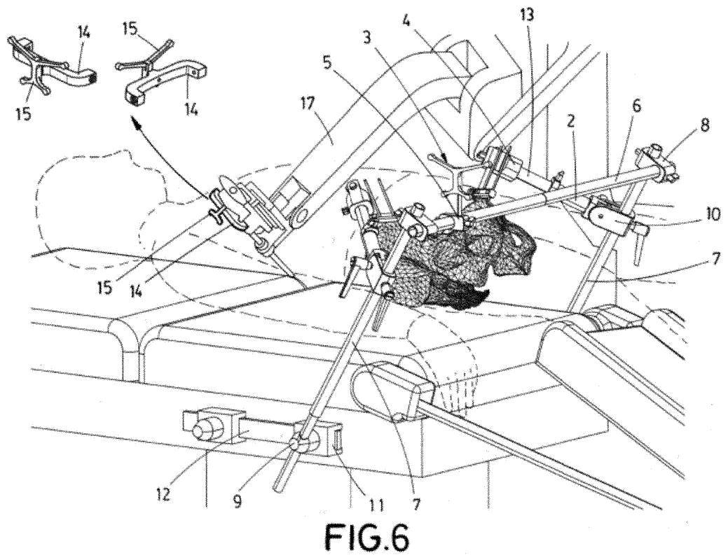

Likewise, the equipment comprises a second reference device (15) configured to be placed in the surgical instrumentation arranged in the robotic arm (17) of the robot by means of a part for fastening to the surgical instrument (14). Said second reference device comprises at least one second reference mark detectable by the navigator of the robot. The first reference mark and the second reference mark enable the spatial position of the first reference device (3) with respect to the anatomy of the patient to be triangulated with the navigator of the robot. FIG. 6 shows a zoomed-in view of the second reference device (15) and the part for fastening to the surgical instrumentation.

As described previously, an essential feature of the invention is that the equipment does not interfere with the scanning equipment. This is thanks to the fact that the fastening arc (1) and the arms (2), with the corresponding fastening means thereof, are made of a radiotransparent material. Preferably, said material is titanium.

Furthermore, in order to ensure a correct fastening of the adapters (4) to the anatomy of the patient, they comprise at least one threaded pin, a screw or another fastening element configured to be anchored to the patient.

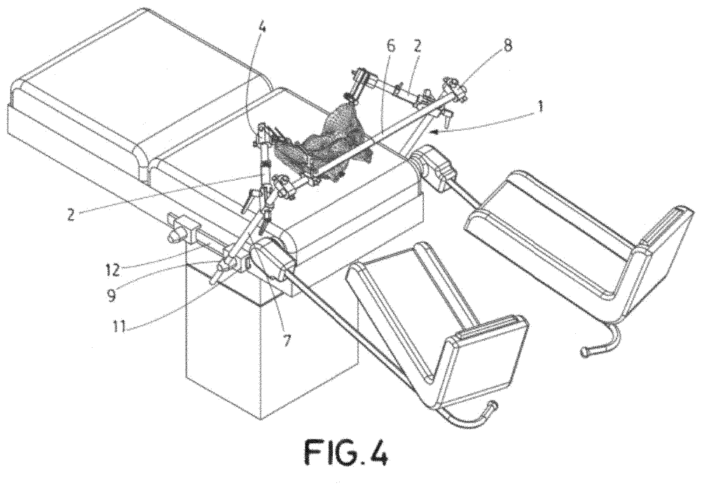

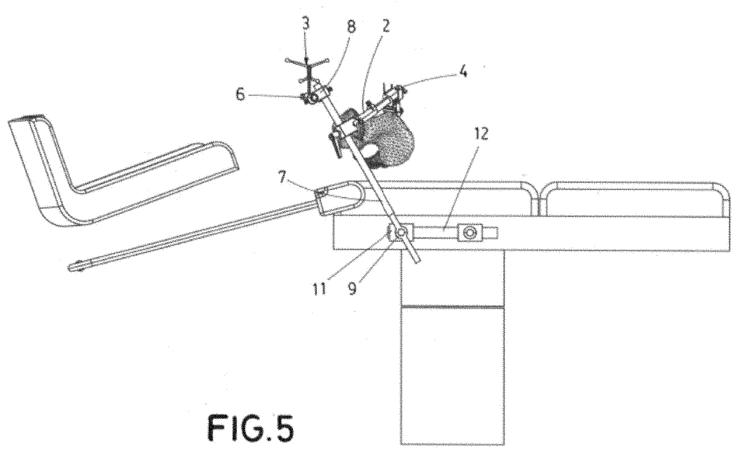

FIGS. 4 and 5 show views of the equipment or a portion of it, perspective and lateral views respectively, as it would be in the final position thereof, joined to the table and to the body of the patient. The high versatility of the equipment can be clearly seen which, thanks to the degrees of freedom of each of the components and fastening means thereof, can be adapted to different table measurements and patient anatomy sizes.

In an exemplary embodiment, like the one shown in said FIG. 5, the second fastening means (9) are mounted on a slider (11) which has the possibility of movement on a rail (12) configured to be joined to the table. Thus, said second fastening means (9) have the possibility of movement on the rail (12).

FIG. 6 shows a perspective view of the equipment and of the robotic arm (17) by means of which the operation is performed. In this case, the hip of the patient to which the adapters (4) of the arms (2) are joined can be seen. In order to facilitate the understanding of the invention, the patient is also represented, by dotted lines, in the position wherein they would be during the operation.

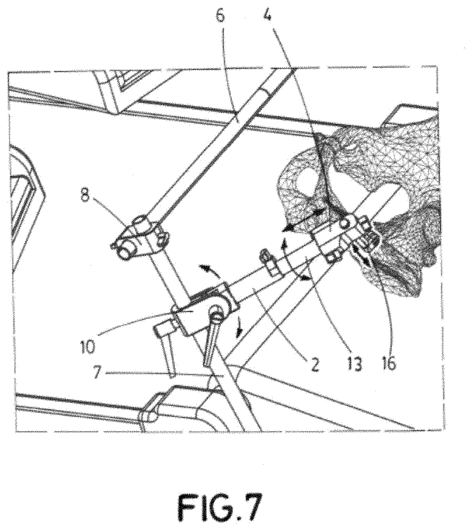

In an exemplary embodiment, the arms (2) can be extended by means of a bar (13) which has the possibility of movement from the lateral bars (7) to determine the length of the arms (2). This embodiment can be seen for example in FIG. 7. Preferably, said bars (13) have the possibility of rotation with respect to the lateral bars (7).

Likewise, also preferably, the adapters (4) can be extended by means of a bolt (16) which has the possibility of movement from the arm (2) in order to determine the length of the adapter (4). In the case of the adapters (4) comprising at least one threaded pin, a screw or another fastening element configured to be anchored to the patient, it is arranged on one end of the bolt (16).

1. Equipment for navigation reference in assisted robotic surgery by means of stereotactic navigation of the organs and soft portions of the pelvic area of a patient with surgical instrumentation installed in a robot, characterised in that it comprises:
a fastening arc (1), made of radiotransparent material, configured to be joined to a surgical table whereon the patient is placed and which in turn comprises:
an upper bar (6) contained in a plane parallel to the surface of the table,
two lateral bars (7) joined to the upper bar (6) forming an arc over the table, by means of first joining means (8) which have the possibility of movement along the lateral bars (7) and the upper bar (6);
second joining means (9) configured to join the lateral bars (7) to the table with the possibility of tilting around said second joining means (9) in order to regulate the inclination of the lateral bars (7) with respect to the table;
two arms (2), made of radiotransparent material, joined to the lateral bars (7) by means of third fastening means (10) which have the possibility of movement along the lateral bars (7), the possibility of rotation with respect to said lateral bars (7) and which comprise a ball joint configured to enable the tilting of the arms (2) with respect to the longitudinal axis of the lateral bars (7), and each arm (2) comprises an adapter (4) on the free end thereof configured to be temporarily anchored to the patient;
a first reference device (3) comprising a mobile part (5) joined to the upper bar (6) with the possibility of movement along it and the possibility of rotation around said upper bar (6) and comprises at least one first reference mark detectable by a navigator of the robot;
a second reference device (15) configured to be placed in the surgical instrumentation of the robot by means of a fastening device in the surgical instrumentation (14) and comprises at least one second reference mark detectable by the navigator of the robot for triangulating the spatial position of the first reference device (3).

2. The equipment according to claim 1, characterised in that the radiotransparent material is titanium.

3. The equipment according to claim 1, characterised in that the second fastening means (9) are mounted on a slider (11) which has the possibility of movement on a rail (12) configured to be joined to the table such that said second fastening means (9) have the possibility of movement on the rail (12).

4. The equipment according to claim 1, characterised in that the arms (2) can be extended by means of a bar (13) which has the possibility of movement from the lateral bars (7) to determine the length of the arms (2).

5. The equipment according to claim 4, characterised in that the bars (13) have the possibility of rotation with respect to the lateral bars (7).

6. The equipment according to claim 1, characterised in that the adapters (4) can be extended by means of a bolt (16) which has the possibility of movement from the arm (2) in order to determine the length of the adapter (4).

7. The equipment according to claim 6, characterised in that the adapters (4) comprise at least one threaded pin, a screw or another fastening element configured to be anchored to the patient.

8. The equipment according to claims 6 and 7, characterised in that the at least one threaded pin, a screw or another fastening element configured to be anchored to the patient is arranged on one end of the bolt (16).
